(19) United States
(12) Patent Application Publication
(10) Pub. No.: US 2015/0142129 A1
(43) Pub. Date: May 21, 2015

(54) Adaptive Stimulation Apparatus and Technique

(71) Applicant: Articulate Labs, Inc., Austin, TX (US)

(72) Inventor: Larry J. Kirn, Austin, TX (US)

(73) Assignee: Articulate Labs, Inc., Austin, TX (US)

(21) Appl. No.: 14/406,559

(22) PCT Filed: Jun. 13, 2013
(86) PCT No.: PCT/US2013/045630

§ 371 Date: Dec. 9, 2014

Related U.S. Application Data
(60) Provisional application No. 61/659,081, filed on Jun. 13, 2012.

(51) Int. Cl. A61F 2/72; A61F 2/80

(52) U.S. Cl. CPC A61F 2/72; A61F 2/80; A61F 2002/701
USPC 623/25

(57) ABSTRACT

In that stimulating muscle within a socket may create additional pressure within the socket that is disrelated to external forces operative on the socket, corruption of pressure measurements within the socket may occur from stimulation, even though negative pressure differential on the side opposing, or furthest from the stimulation area, is explicitly used so as to avoid this corruption. An embodiment of the invention provides removal from the total pressure measurement that portion which is known to be resultant of muscle contraction. This stabilizes overall system control through improving input signal quality. Other embodiments are described herein.

### BACKGROUND

The preferred interface method between prosthetic limbs and residual skeletal members is a socket which encloses the residual limb. These sockets are typically of a hard inflexible material, such as carbon fiber or fiberglass, with distal attachment to a prosthetic hand, foot, etc. Hard construction in this manner distributes force relatively evenly over the enclosed residual limb, and readily accommodates incident forces without undue flexion, improving user's spatial prediction while moving with the prosthesis.

Fabrication of well-performing prosthetic sockets is a costly, individualized proposition that remains much more an art than a science. Once a new socket is shown to perform well for a given patient, every effort possible is made to keep that socket in use for the longest term possible. However, fluctuations in residual limb volume, which occur naturally with variance of patient activity and weight, frustrate that goal. In that commonly-used prosthetic sockets have no capacity to change internal volume, the preferred treatment modality is currently to encourage atrophy of the muscle which is to be contained within the socket, prior to fitting a definitive socket. This approach leads to a relatively constant contained volume which is less affected by external conditions than active limbs, prolonging the useful lifetime of a socket. The consistency of atrophied tissue furthermore tends to behave in an isotropic manner, translating axial force into distributed compressive force against the socket wall.

Allowing contained muscle to atrophy, however, compromises several fundamental aspects of prosthetic use. Skin surrounding the residual musculature tends not to atrophy with the muscle, leaving in many cases a large amount of excess tissue. Fat distribution tends to increase in dormant muscle, further increasing tissue compliance that impairs prosthetic limb control. Ability of residual muscle to cushion bone from the enclosing socket diminishes directly with muscle mass, possibly leading to painful collisions of unprotected bone and prosthetic socket with every step.

A flaccid tissue mass surrounding bone, resulting from intentional muscle wasting, usually fails utterly to firmly couple socket position to the enclosed skeletal structure.

The consequence for poor skeletal coupling in upper-limb prostheses is usually localized skin injury such as rashes and lesions; in lower-limb prostheses, the higher tissue volume often additionally results in poor spatial prediction, unreliable position control, and severely impaired proprioception—an individual's unconscious sense of limb location. These factors are key contributors to a much higher incidence of falls in lower-limb amputees.

In that limb prosthetic use most often results from amputation, significant impairments from amputation itself are imposed upon, and may easily be exacerbated by, prosthetic use. Transection of peripheral nerves often results in neuroma formation and phantom pain. While nerve termination within active tissue such as muscle has shown benefit, successful palliative measures against neurological consequences of amputation remain elusive. Circulation through the residual limb is severely impaired by an amputation, a situation often compounded by the fact that most amputations are prompted by vascular deficiency. Secondary to neurological and/or circulatory impediments, thermal regulation in residual limbs is usually seriously impaired, very often exacerbating phantom and stump pain.

A fundamental element common to many difficulties with prosthetic sockets is that of directional control. Simple firing of an internal muscle mass, no matter how well-timed, is incapable of addressing both physical and biological demands imposed by use of a prosthesis.

### BRIEF DESCRIPTION OF THE DRAWINGS

Embodiments of the present invention will be described by way of exemplary embodiments, but not limitations, illustrated in the accompanying drawings in which like references denote similar elements, and in which:

FIG. 1 shows an axial cross-section view of a prosthetic socket into which an exemplary embodiment of the present invention has been incorporated, as fit over a human limb, such as a leg.

FIG. 2 shows an anterior aspect view of the prosthetic socket of FIG. 1.

FIG. 3 shows a simplified control block diagram of the embodiment of FIGS. 1 and 2.

FIG. 4 shows input and output waveforms of the control system of FIG. 3.

FIG. 5 shows an alternative control block diagram of the embodiment of FIGS. 1 and 2.

FIG. 6 shows commonly-accepted phases of the human gait cycle.

FIG. 7 shows output waveforms of an embodiment of the invention in relation to the gait cycle of FIG. 6.

FIG. 8 shows an electrode assembly for use with an embodiment of the invention.

FIG. 9 shows an alternative electrode assembly for use with an embodiment of the invention.

FIG. 10 shows an alternative prosthetic liner fabrication suitable for use with an embodiment of the invention.

FIG. 11 shows a block diagram of signal processing which may be performed within or by Controller 509 of FIG. 5 in an embodiment of the invention.

FIG. 12 includes a system for implementing the controller and other aspects of various embodiments of the invention.

### DETAILED DESCRIPTION

Various operations will be described as multiple discrete operations, in turn, in a manner that is most helpful in understanding the illustrative embodiments; however, the order of description should not be construed as to imply that these operations are necessarily order dependent. In particular, these operations need not be performed in the order of presentation. Further, descriptions of operations as separate operations should not be construed as requiring that the operations be necessarily performed independently and/or by separate entities. Descriptions of entities and/or modules as separate modules should likewise not be construed as requiring that the modules be separate and/or perform separate operations. In various embodiments, illustrated and/or described operations, entities, data, and/or modules may be merged, broken into further sub-parts, and/or omitted. The phrase “embodiment” is used repeatedly. The phrase generally does not refer to the same embodiment; however, it may. The terms “comprising,” “having,” and “including” are synonymous, unless the context dictates otherwise. The phrase “NB” means “A or B.” The phrase “A and/or B” means “(A), (B), or (A and B).” The phrase “at least one of A, B and C” means “(A), (B), (C), (A and B), (A and C), (B and C) or (A, B and C).”

An embodiment takes advantage of multiple contained muscles. More specifically, the embodiment includes selective stimulation of individualized residual musculature to provide a generalized solution which does not require extensive customization for each patient.

An embodiment provides directional control of a prosthetic socket via dynamic selective stimulation of enclosed musculature, while mitigating muscular, circulatory, and neurological impacts of prosthetic use.

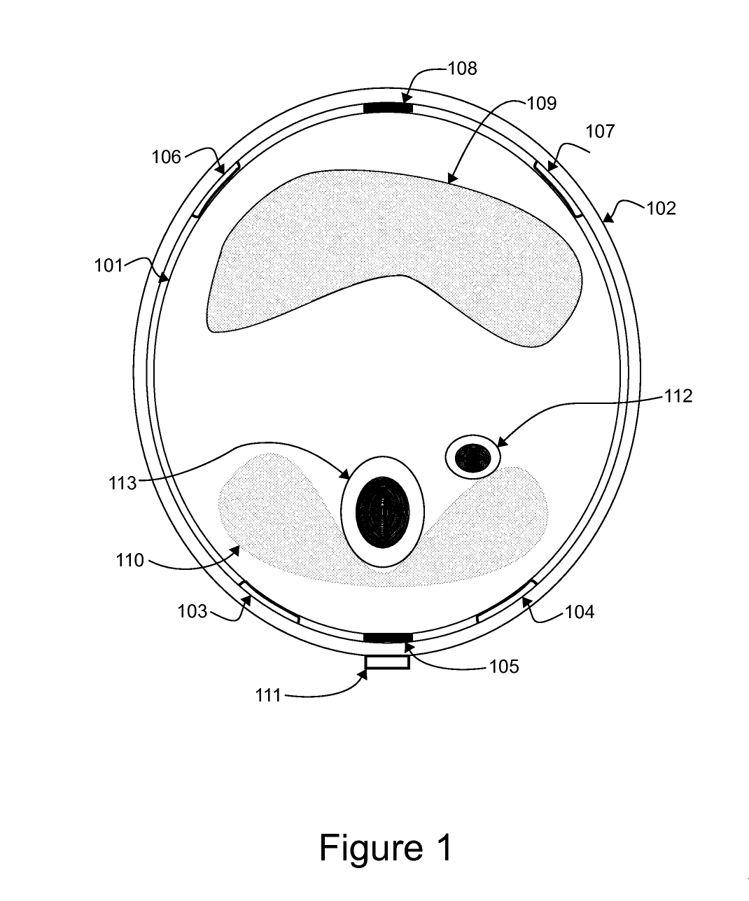

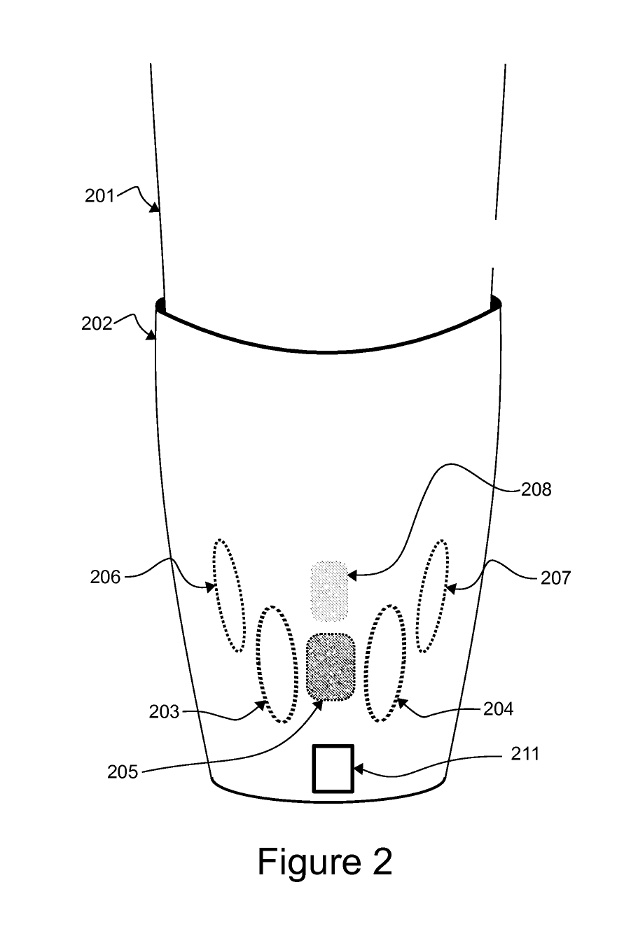

FIG. 1 projects on the axis of a limb in a distal direction. FIG. 2, truncated for brevity on both proximal and distal ends, projects perpendicularly to the axis of the limb, presumably from the anterior aspect. Note that simplification or idealization of Limb 101, done for illustrative purpose, may introduce deviations from commonly encountered anatomy, which are not directly relevant to the present invention.

Referring now to FIG. 1, Prosthetic Socket 102 encloses residual Limb 101, which may be any body member to which a prosthetic socket may be affixed. Bones 112 and 113 are assumed to be truncated at the distal end. Muscle Masses 109 and 110 consist of muscle tissue within the posterior and anterior portions, respectively, of Limb 101. Sensors 105 and 108 provide signals indicative of anterior and posterior pressure, respectively, between Limb 101 and the interior surface of Socket 102. Sensors 105 and 108 may optionally be possessed of bandwidth adequate to provide signals indicative of sonic or vibrational activity within Limb 101, to ultimately measure contractile force of underlying muscle. For example, frequencies from 0 to approximately 20 Hz. may impart salient information about internal pressure between residual limb tissue and the socket, while frequencies from 0.5 Hz. To 200 Hz. may provide more than adequate information to infer muscle contractile force. Measurement of muscle contractile force by detecting resultant acoustic emissions is known in the art as acoustic myography or vibromyography, and may serve to improve prosthesis control by indicating which portion of localized internal socket pressure is resultant of muscle contraction, in contrast to internal pressures resultant of external forces applied to the socket. While direct output of a pressure sensor may suffice to determine pressures (or lack thereof) within a socket; techniques such as are described in U.S. patent application Ser. No. 12/759,344 ‘Acoustic Myography System and Methods’ may be employed in order to obtain high-quality signals indicating muscle contractile force.

Stimulation Electrodes 103 and 104 are affixed within Socket 102 in contact with the skin of Limb 101, so as to facilitate stimulation of underlying muscle Mass 110. Electrodes 106 and 107 are similarly positioned on the posterior internal surface of Socket 102, so as to facilitate stimulation of underlying muscle Mass 109. An elastomeric liner, possibly of a material such as urethane or silicone, may be used between Limb 101 and Socket 102, the only constraint being that conductivity be maintained between Electrodes 103, 104, 106, and 107 and the skin of Limb 101. Optional three-axis Accelerometer 111 provides inertial signals indicative of motion of the prosthesis through space, to improve integration of prosthesis control with the user's gait.

Sensor 105 may employ any technique to measure static compressive force, such as carbon-filled ink laminated within film surfaces or load cells; or may be of a piezoelectric material capable of higher-frequency response, such as shielded silver-inked piezoelectric film. In use, it can be seen that Sensor 105 will provide at least force information of Limb 101 (practical with both low-frequency and higher-frequency devices) and optionally contractile force of muscle Mass 110 (if implemented as a higher-frequency device such as a piezoelectric sensor), both against the anterior inner surface of Socket 102. Determination between internal pressure and muscle contractile force may be made by signal processing circuitry or software executed by Processor 309 (below) in receipt of Sensor 105 output, following techniques known to the art. Sensor 108 will similarly provide force information of Limb 101 and optionally contractile force of muscle Mass 109, both against the posterior inner surface of Socket 102. In that Limb 101 is at least partially comprised of compliant tissue, common-mode pressure signals between Sensors 105 and 108 will be associated with axial force on Limb 101. Differential pressure signals between Sensors 105 and 108, however, will be associated with forces in the anterior-posterior plane, or Y-axis of FIG. 1. Especially under conditions of light axial limb loading, higher socket forces in anterior or posterior directions can therefore be anticipated to cause posterior or anterior limb pressures, respectively, to go to zero as Limb 101 loses contact with Socket 102.

Referring now to FIG. 2, Leg 201, Socket 202, Stimulation Electrodes 203, 204, 206, and 207, Sensors 205 and 208, and Accelerometer 211 refer directly to similarly numbered Leg 101, Socket 102, Stimulation Electrodes 103, 104, 106, and 107, Sensors 105 and 108, and Accelerometer 211, respectively, all of FIG. 1. Accelerometer 211, although shown to be mounted on the exterior surface of Socket 202, may be affixed to or integrated with Socket 202 at any advantageous location, the sole constraint being that Accelerometer 211 indicate spatial dynamics of Socket 202. Stimulation Electrodes 203 and 204, as well as Sensor 205, are affixed to the anterior (near) interior surface of Socket 202. Stimulation Electrodes 206 and 207, as well as Sensor 208, are affixed to the posterior (far) interior surface of Socket 202. Thus, FIG. 2 is not meant to imply 203, 204, 205, 206, 207, and 208 are all on the anterior or posterior inside surface of the socket.

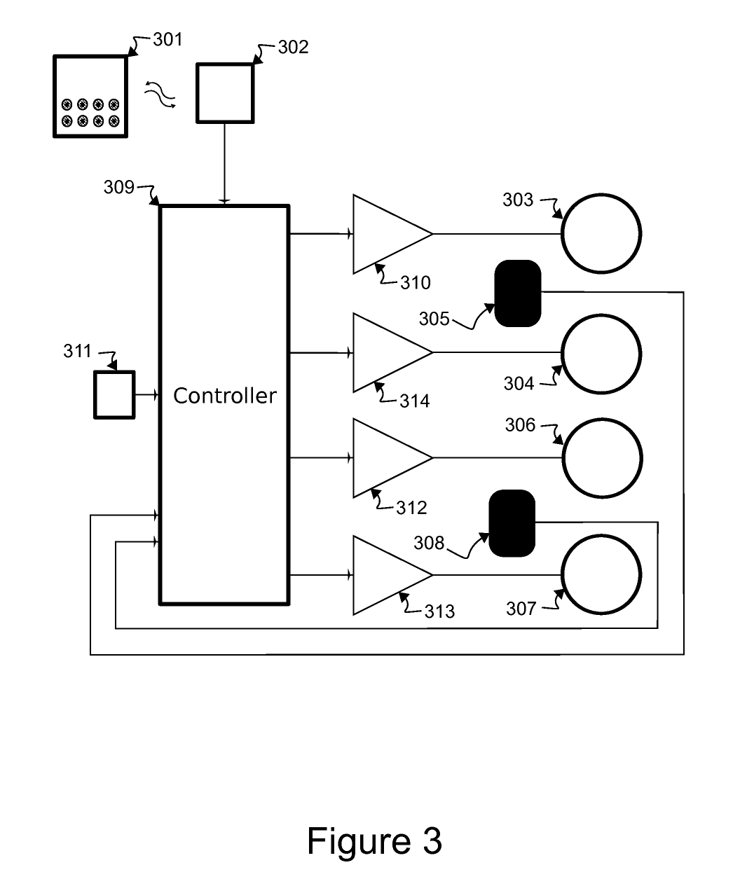

Referring now to FIG. 3, Accelerometer 311 and Sensors 305 and 308 correspond to Accelerometer 111 and Sensors 105 and 108, respectively, all of FIG. 1. Electrodes 303, 304, 306, and 307 as well correspond to Electrodes 103, 104, 106, and 107, respectively, all of FIG. 1. Optional Accelerometer 311 provides information regarding position and motion in space of prosthetic Socket 102 of FIG. 1 to Controller 309. Sensors 305 and 308 provide localized information regarding internal pressures and optionally sonic activity within Socket 102 of FIG. 1 to Controller 309.

Remote Interface 302 provides wireless connectivity between Remote Control Device 301 and Controller 309. Said connectivity may be through any means, such as radio frequency, infrared, inductive coupling, etc. Remote Control Device 301 may be implemented as a stand-alone device such as an RF key fob, a more intelligent device such as a smart phone, a computer, or any other device suitable for human control and/or monitoring of the invention. Connectivity between Device 301 and Interface 302 may be bidirectional or unidirectional in either direction. Use of Control Device 301 may be to control embodiments of the invention, obtain information regarding use or dynamics of the invention or wearer, or any other required interactivity.

Through execution of algorithms described herein, Controller 309 emits control signals to constant-current Amplifiers 310, 314, 312, and 313, which in turn apply controlled constant-currents to Electrodes 303, 304, 306, and 307, respectively. In that Electrodes 103 and 104 are positioned to stimulate muscle Mass 110, and Electrodes 106 and 107 are positioned to stimulate muscle Mass 109, all of FIG. 1; differential currents will preferentially be simultaneously applied across the pair consisting of Electrodes 303 and 304, and the pair consisting of Electrodes 306 and 307 (i.e., some portion of current between electrodes 103, 104 will be delivered at the same time as some portion of current between electrodes 106, 107. Alternative application of stimulation potentials and/or currents, however, do not escape the scope of the invention, nor does muscle stimulation through other means, such as magnetic stimulation. In response to information from Sensors 305 and 308 and optional Accelerometer 311, Controller 309 can be seen to independently control stimulation of muscle Masses 109 and 110 of FIG. 1.

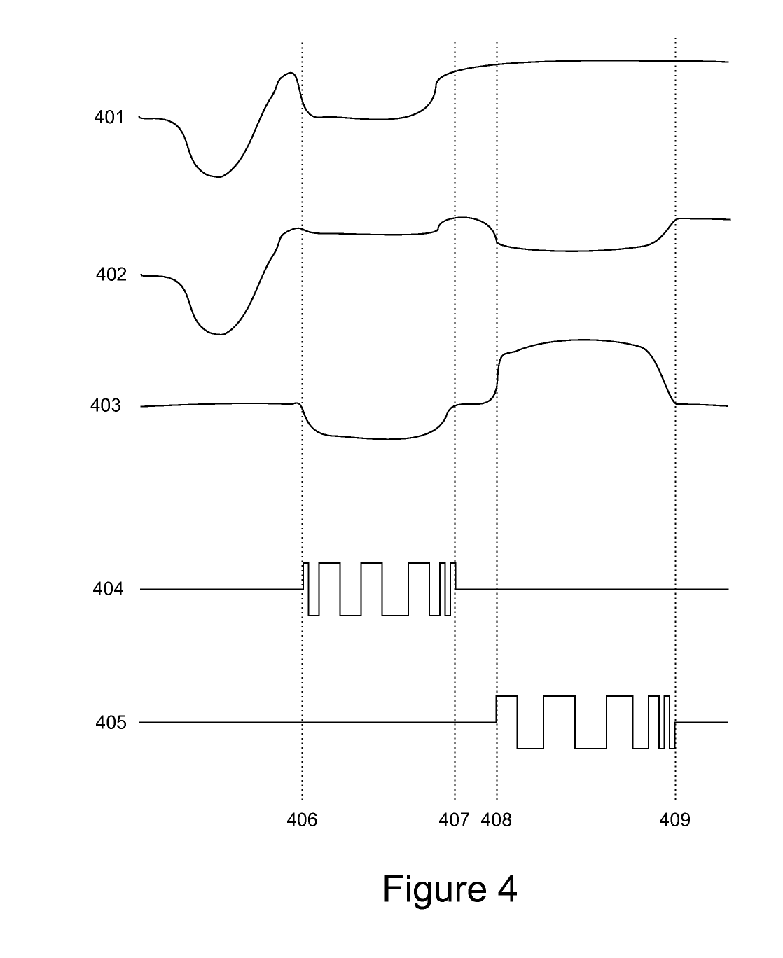

Referring now to FIG. 4, Force Waveforms 401 and 402 show absolute internal force or pressure between Limb 101 and Socket 102 present at Sensors 105 and 108, respectively, all of FIG. 1. Differential Force 403 shows the difference between Force 401 and 402 with a gain factor of approximately two. Differential Stimulation Outputs 404 and 405 show differential output currents applied by an embodiment of the invention between Electrodes 106 and 107, and Electrodes 103 and 104, respectively, again all of FIG. 1. Differential currents supplied between Electrodes 106 and 107; and Electrodes 103 and 104 may derive from any output stage topology, such as single-ended outputs referenced to a common ground, or bridge-tied load. Although minimal DC offset is shown in Outputs 404 and 405, use of DC offsets does not escape the scope of the invention.

The X-axis of FIG. 4 is time; Markers 406, 407, 408, and 409 are demarcations of salient physical events as a wearer of Socket 102 of FIG. 1 uses the prosthesis to perform a physical action. For example, in the event that Socket 102 of FIG. 1 encloses a lower leg, Force Waveforms 401, 402, and 403 of FIG. 4 may be resultant of the wearer taking a stride with the prosthetic leg.

Before Time Marker 406, Force Waveforms 401 and 402 show similar dynamic force measurements, resulting in minimal differential force as shown in Waveform 403. Waveform 403 before Marker 406 therefore indicates minimal differential force between Sensors 105 and 108 of FIG. 1. Dynamic common-mode activity of this nature is to be expected when axial compressive load is placed on a prosthesis with no anterior-posterior force component. At Marker 406, however, Waveforms 401, 402, and 403 show that pressure against anterior Sensor 105 becomes significantly less than that against Sensor 108, both of FIG. 1. Differential force of this nature is associated with external application of anterior-posterior force by the wearer of the invention. For the duration that internal socket forces are in this condition, Output 404 shows differential stimulation application between Electrodes 106 and 107 of FIG. 1. The resultant contraction of muscle Mass 109 of FIG. 1 exerts addition force against Sensor 105, arresting further negative travel in Waveform 401 and 403.

At Time Marker 407, differential force shown in Waveform 403 again returns to a low value, presumably caused by anterior-posterior force cessation by the wearer of the invention. Resultantly, stimulation current shown in Output 404 ceases.

Following to the right at Marker 408, Force Waveforms 401, 402, and 403 show that pressure against anterior Sensor 108 becomes significantly less than that against Sensor 105, both of FIG. 1. This represents application of anterior-posterior force by the wearer in the opposing direction to that shown at Marker 406. For the duration of this condition, Output 405 shows differential stimulation application between Electrodes 103 and 104 of FIG. 1. The resultant contraction of muscle Mass 110 of FIG. 1 exerts additional force against Sensor 108, arresting further negative travel in Waveform 402 and 403. At Marker 409, reduction of differential force seen in Waveform 403 can again be seen to cause reduction of Output 105 to zero.

Note that current pulse widths shown in Outputs 404 and 405 increase as differential force decreases on the opposing sensor. Operating on principles noted in the following paragraph, this represents a proportional closed-loop system, wherein pulse period is directly proportional to negative differential at the sensor opposing a stimulation electrode pair. The relatively flat regions in Differential Force 403 between Markers 406 and 407, and between Markers 408 and 409 are resultant of proportionally-controlled stimulation with control loop gain less than infinity.

Muscles exhibit peak contractile force at frequencies approximately between 2 kHz and 3 kHz, but higher frequencies, such as 20 kHz, predispose cell to fire more readily. Afferent nerve sensitivity as well falls with increasing frequency, reducing sensation perceived by the wearer at higher stimulation frequencies. Increase of stimulation current pulse period, which increases its frequency, in step with increasing stimulation demand therefore improves overall response time to rapid changes in desired contractile force. Intensity control through pulse period therefore improves transient response of an embodiment of the invention over conventional approaches, such as dynamically changing the stimulation current of a constant pulse period applied.

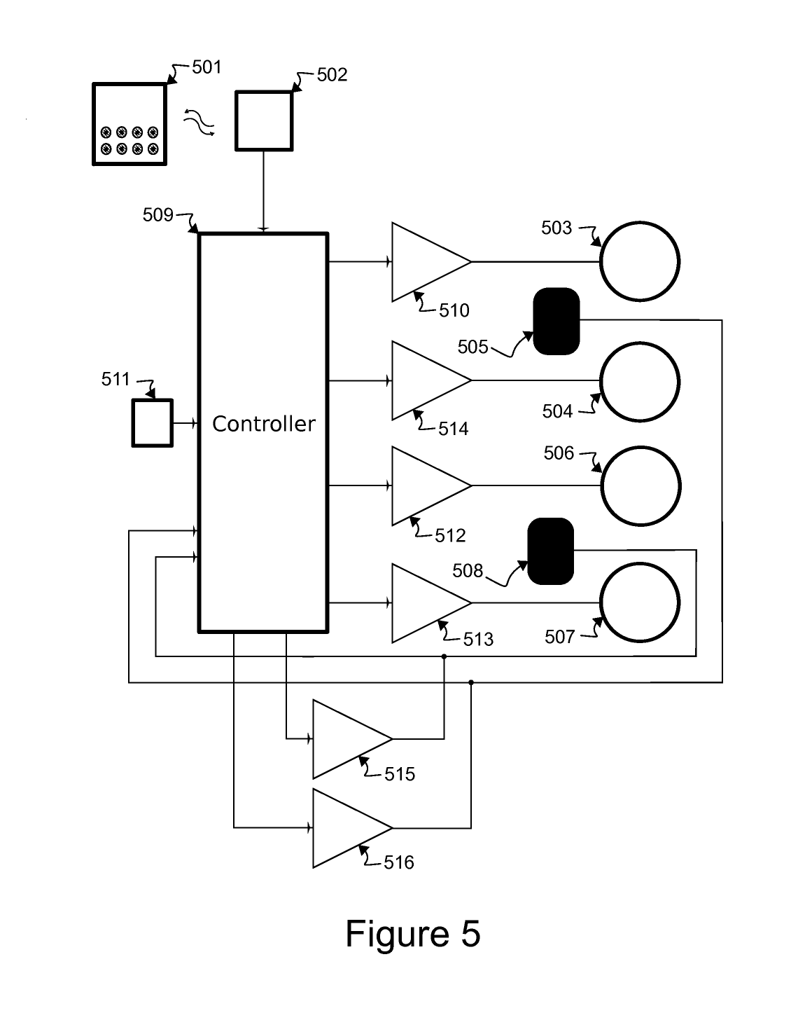

Referring now to FIG. 5, Remote Control Device 501, Remote Interface 502, Electrodes 503, 504, 506, and 507, Controller 509, and Amplifiers 510, 514, 512, and 513 correspond to Remote Control Device 301, Remote Interface 302, Electrodes 303, 304, 306, and 307, Controller 309, and Amplifiers 310, 314, 312, and 313, respectively, all of FIG. 3. Sensors 505 and 508, which physically correspond to Sensors 305 and 308, respectively, of FIG. 3, are of a material exhibiting piezoelectric properties. Being in contact with enclosed Limb 101 of FIG. 1, Sensors 505 and 508 therefore provide information to Controller 509 regarding both dynamic force and sonic or vibrational activity within Limb 101 of FIG. 1.

Under control of Controller 509, Constant-current Amplifiers 515 and 516 may inject current into the outputs of Sensors 508 and 505, respectively. The impedance of piezoelectric materials is known to change with physical load. During times that Controller 509 commands current injection into these sensor outputs, the voltages presented by Sensors 505 and 508 to Controller 509 are therefore inversely proportional to respective sensor impedances, and hence indicative of the physical pressures imposed on Sensors 505 and 508. During times that Controller 509 does not command current injection into these sensor outputs, the voltages presented by Sensors 505 and 508 to Controller 509 will then convey acoustic emissions of the underlying muscle, acting as a contact microphone. For example, current injection may be commanded by Controller 509 for a few milliseconds every 20 milliseconds, to obtain socket pressure information, and command no injection at all other times, so as to receive muscle acoustic emissions. Sensors 505 and 508 may be of any material exhibiting piezoelectric properties, such as ceramic or polarized plastic film, which is amenable to lamination within a prosthetic socket. Excitation current may be provided to Sensors 505 and 508 at a frequency at least twice the highest frequency of muscle sonic emissions to be measured (following Nyquist's theorem); or may employ pulsed excitation, wherein rates of voltage change are used to infer transducer impedance. Detection of sensor impedance while under AC excitation may be accomplished through rectification and/or peak detection of the resultant voltage signals at the sensor inputs. Detection of sensor impedance while under pulsed excitation may be accomplished through measuring rates of change of the resultant voltage signals at the sensor inputs. For periods during which excitation is not present, Sensors 505 and 508 may provide signals representative of muscle acoustic emissions, similarly to any other microphone. Through use of this technique, a single simple transducer may be used to provide both pressure and acoustic conditions of an enclosed limb.

In that prosthetics address both biological and mechanical requirements, recruitment of residual muscle into a synergistic relationship with a socket must as well address both biological and mechanical constraints. For example, while firing muscle continuously may bolster prosthetic rigidity, it would ultimately result in tissue damage. Conversely, use of a fixed stimulation program with no regard to physical use of the prosthesis would introduce physical instability in the prosthesis. Balance of these two requirements is highly individualized, and is furthermore reliant on specific activities or activity levels.

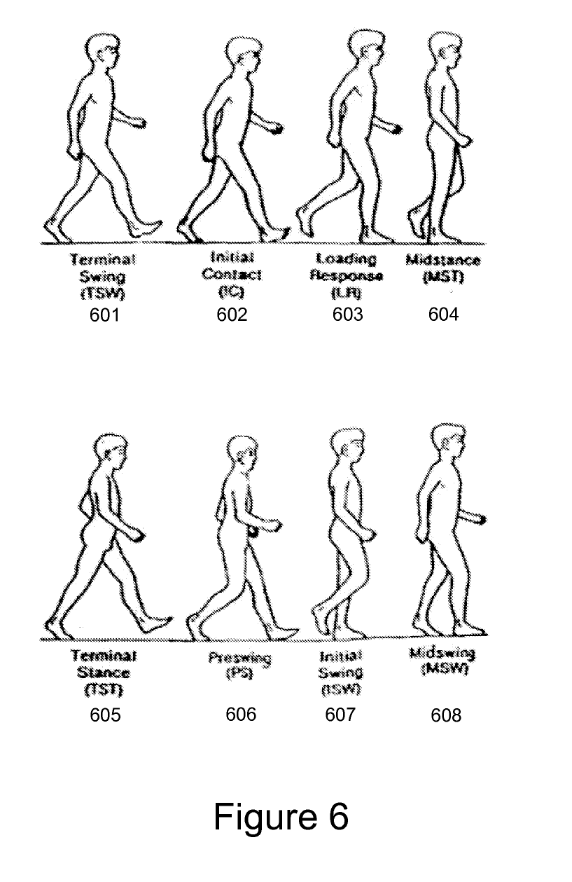

Referring now to FIG. 6, a human is shown as the right leg progresses through eight commonly accepted phases of gait—Terminal Swing 601, being immediately before planting a foot (known as heel strike), Initial Contact 602, wherein the heel first strikes the surface, Loading Response 603, wherein forward propulsion commences, Midstance 604, at which point maximal gravitic force exists, Terminal Stance 605, wherein forward propulsion culminates through use of calf muscles, Preswing 606, wherein forward propulsion terminates, Initial Swing 607, at which point the foot leaves the surface, and Midswing 608, wherein the knee begins to straighten in preparation for the following heel strike.

In the event that a lower-limb prosthetic is used on the active leg, it can be seen that axial prosthetic force, or compressive force along the axis of the leg, will exist through Phases 602, 603, 604, 605, and 606; and that anterior-posterior prosthetic force, or force in the sagittal plane resultant of the leg being used to propel the body forward, will exist through Phases 603, 604, 605, and 606. Gait analysis using force instrumentation has repeatedly shown propulsive force to be highest in Loading Response 603 and particularly Terminal Stance 605, following rates of change in forward velocity at these points of the gait cycle. Due to lack of axial loading, prosthesis control by the wearer is reduced in Phases 607, 608, and 601.

In an intact limb, plantarflexion, or rotation of the foot to move the toes in a downward direction, of the foot under control of the gastrocnemius begins at Phase 604, culminating to maximum contractile force at Phase 605. Dorsiflextion, or rotation of the foot to raise to toes, of the foot, under primary control of the tibialis anterior begins at Phases 607 and terminates at Phase 608.

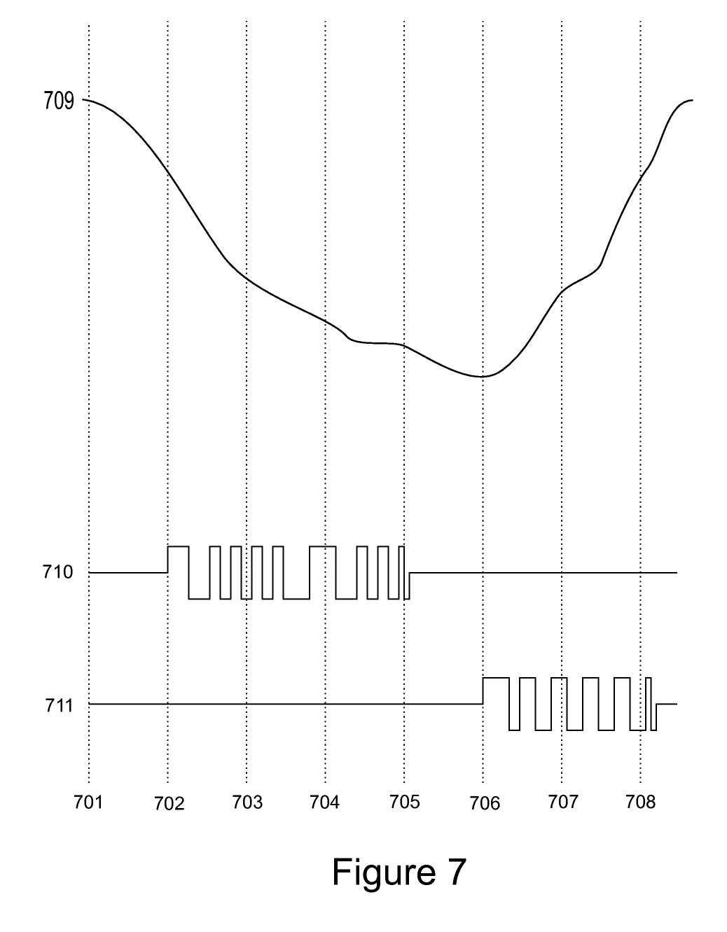

Referring now to FIG. 7, Position 709 shows sagittal plane orientation of the active leg tibia traversing the gait phases given in FIG. 6. Increasing value of Position 709 indicates anterior inclination, decreasing value indicates posterior inclination. Sagittal position is presumably calculated by Controller 309 in response to Accelerometer 311, both of FIG. 3, with increasing value indicating anterior inclination. Outputs 710 and 711 show differential stimulation currents applied by an embodiment of the invention to the anterior Electrode pair 103 and 104, and posterior Electrode pair 106 and 107, respectively, all of FIG. 1. Markers 701, 702, 703, 704, 705, 706, 707, and 708 correspond to gait Phases 601, 602, 603, 604, 605, 606, 607, and 608, respectively, all of FIG. 6. Although the X-axis represents time, relative phase timing shown does not necessarily reflect normal human gait. Waveforms of FIG. 7 show operation of an embodiment of the invention when implemented on a right (active) leg prosthetic socket on the human figure of FIG. 6.

At Terminal Swing 701, posterior sagittal movement begins, shown by initial downward travel (indicative of posterior movement) in Position 709, as the foot proceeds toward heel strike. At Initial Contact, air (if any) contained within the prosthetic socket will begin to be expelled by axial compressive force. A one-way valve installed in the distal portion of the socket is commonly used for this purpose. At Loading Response 703, the majority of air will be expelled, and stimulation of the posterior calf muscle begins, as shown in Output 710. Contraction of posterior muscle at Phase 703 serves to stabilize the socket against the high propulsive force applied at this phase. Stimulation of posterior muscle continues through Midstance 704, Terminal Stance 705, and Preswing 706.

Note that the pulse periods shown in Output 710 directly reflect the higher propulsive force experienced at Phases 703 and 705. Increase in pulse period is directly proportional to sagittal acceleration of the leg, being calculated by Controller 309 in response to input from Accelerometer 311, both of FIG. 3. Note also that posterior muscle stimulation shown in Output 710 does not terminate until Preswing 706. Continuation of stimulation past the point of propulsive force serves to disallow air ingress into the socket as axial force plummets. The combination of no stimulation at Phase 702 (to allow air to be expelled from the socket) with stimulation through Phase 706 (to retain firm socket contact so as to retain socket vacuum) forms a pumping cycle to retain socket vacuum. Note that stimulation of posterior muscle is only slightly modified, perhaps by less than 100 milliseconds, from normal gastrocnemius activity in an intact limb noted above, with minimal deviation from the proportional control loop Output 404 of FIG. 4.

At Preswing 706, anterior stimulation begins, as seen in Output 711. High initial period shown is to offset the high physical hysteresis of the socket as sagittal direction changes, and as well protect against air ingress. Anterior stimulation can be seen to continue in Output 711 through Initial Preswing 707 and Midswing 708, until sagittal motion is arrested, as seen in Position 709. Note that stimulation of anterior muscle is again only slightly modified, perhaps by less than 200 milliseconds, from normal tibialis anterior activity in an intact limb noted above, with minimal deviation from the proportional control loop Output 405 of FIG. 4.

It is assumed that the conditions indicating specific gait cycle phases at which time specific muscle areas are to be stimulated are dynamically identified by the wearer or a health professional, preferably using Remote Control Device 501 of FIG. 5, and that these conditions are stored by Controller 509 of FIG. 5 for subsequent identification of these specific gait cycle phases.

A minimum contractile duration is required both for reaction against a socket, and to induce blood flow. A minimum period of stimulation, such as 200 to 500 milliseconds, is therefore assumed to be enforced upon every stimulation event. Continuous muscular contraction, however, severely attenuates blood flow. For example, continuous stimulation for longer than 30 seconds has been shown to shown to dramatically increase fatigue through oxygen starvation in the muscle. A minimum refractory period, such as 200 to 400 milliseconds, between stimulation events is therefore also assumed to be enforced. Embodiments of the invention as described above perform no action during periods of inactivity. In order to provide relatively continuous assistance to blood flow, periodic stimulation events are to be optionally provided by an embodiment of the invention during periods of prosthetic inactivity.

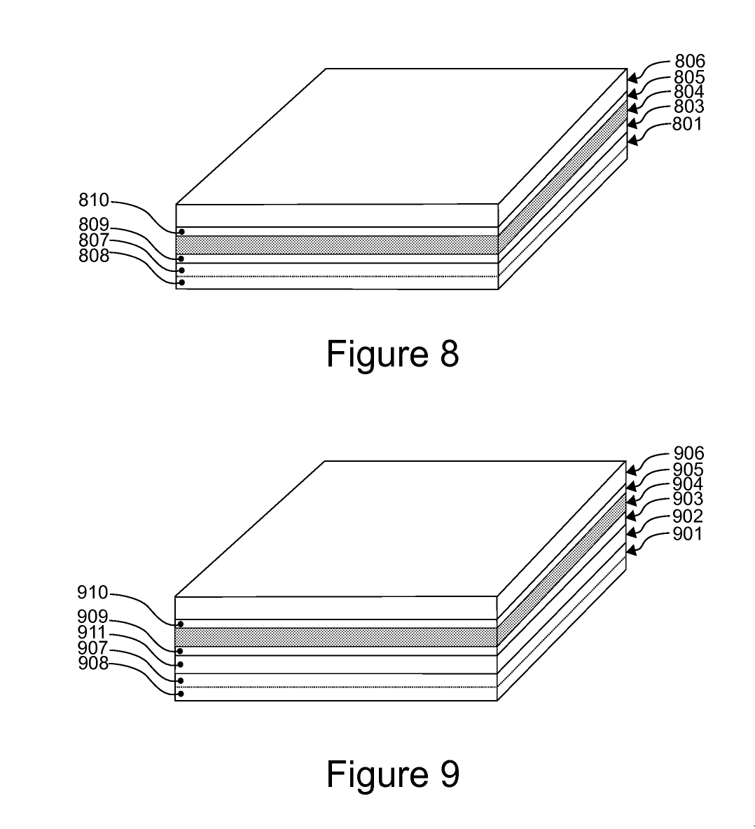

Referring now to FIG. 8, the lower surface of Force Transducer 801 is to be directly laminated to or otherwise secured to either the interior surface of a prosthetic socket or the inner surface of a prosthetic liner for use with a socket. Transducer 801 may employ any technique to measure static compressive force, such as carbon-filled ink laminated within film surfaces, or may be of a piezoelectric material, such as shielded silver-inked piezoelectric film. It is assumed that exterior faces of Transducer 801 are electrically non-conductive. Conductors 807 and 808 are electrically connected to Transducer 801 and serve to carry force information to measurement means, such as Controller 309 of FIG. 3. On the upper Transducer 801 surface away from the socket or liner, Shield 803 is laminated, and connected by Conductor 809 to a ground reference, such as may be expected within Controller 309 of FIG. 3. Shield 803 serves to limit noise ingress into Transducer 801. On the upper Shield 803 surface away from the socket or liner, Insulator 804 is laminated, serving to electrically isolate Shield 803 from upper layers. On the upper Insulator 804 surface away from the socket or liner, low-resistance Conductive Substrate 805 is laminated and electrically connected by conductor 810 to drive means, such as Amplifier 310, 314, 312, or 313 of FIG. 3. In the event that a rigid electrode is acceptable, optional Conductive Layer 806 is not used and Substrate 805 is of material suitable for long-term skin contact, such as stainless steel 316L. In the event that a flexible electrode is required, Conductive Layer 806 is laminated on the upper surface of Substrate 805 away from the socket or liner. Conductive Layer 806 may be of any flexible material which conducts electricity and is suitable for long-term skin contact, such as carbon-filled silicone. In that materials of this nature usually exhibit relatively high resistance, Substrate 805 may be smaller than Conductive Layer 806, so as to progressively limit current at the edges of the assembly.

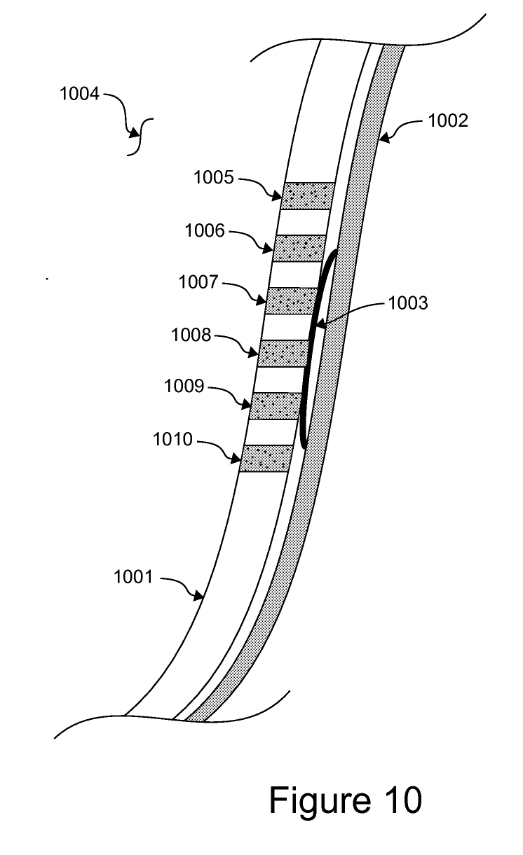

Referring now to FIG. 9, Transducer 901, Shield 903, Insulator 904, Substrate 905, Conductive Layer 906, and Conductors 907, 908, 909, 910, and 911 correspond to Transducer 801, Shield 803, Insulator 804, Substrate 805, Conductive Layer 806, and Conductors 807, 808, 809, 810, and 811, respectively, all of FIG. 8. Piezoelectric Layer 903 is laminated between Transducer 901 and Shield 903, and provides a signal denoting sonic or vibrational information through Conductor 911 to measuring means, such as Controller 309 of FIG. 3. The assembly of FIG. 9 is preferred over that of FIG. 8 in the event that both piezoelectric and static force transducers are to be employed with an embodiment of the invention.

In use, the assembly\ies of FIGS. 8 and 9 may be used at locations noted for Electrodes 103 and 104, as well as Electrodes 106 and 107, all of FIG. 1. Common signal between transducer assemblies at Electrode 103 and 104 positions, for example, may indicate average pressure existing at the physical point between the two electrodes, or at the position indicated for Sensor 105 of FIG. 1. As can be seen in FIGS. 8 and 9, pressure sensing may be accomplished over the entire surface of a stimulation electrode, significantly reducing the area of individual sensors and electrodes.

Referring now to FIG. 10, prosthetic Liner 1001 is worn over a residual Limb 1004, inside a prosthetic Socket 1002. Liner 1001 preferably is of elastomeric material, such as urethane or silicone, so as to provide cushion between Limb 1004 and Socket 1002. Electrode 1003 is affixed to the interior surface of Socket 1002 and presumably electrically connected to circuitry requiring contact with the skin of Limb 1004. Prosthetic Liner 1001 represents one possible addition to an embodiment of the invention wherein a prosthetic liner is necessary between Leg 101 and Socket 102 of FIG. 2.

As is common practice, Liner 1001 is presumed to be cast in a mold into which uncured elastomeric material is injected. In areas of Liner 1001 which will possibly require electrical contact with Limb 1004, spatially periodic injections of elastomer filled with conductive material are performed as part of the injection process. These injection sites result in shown Vias 1005, 1006, 1007, 1008, 1009, and 1010. Adequate conductive filler, such as carbon black, carbon nanotubes, or silver-plated copper beads, is to be used so as to achieve a percolation limit conducive to relatively low electrical resistance. For the purposes of electrical muscle stimulation, which may involve currents around 200 milliamps, resistance between the driving current amplifier and the electrode surface will preferably be below that to cause a low voltage drop, such as 2-5 volts.

In use, liner Vias, such as 1005, 1006, 1007, 1008, 1009, and 1010 provide electrical conduction between the skin of Limb 1004 and Electrode 1003. Note that position of electrodes relative to Limb 1004 is physically determined by electrode placement within Socket 1002, and not affected whatsoever by positioning of Liner 1001 on Limb 1004.

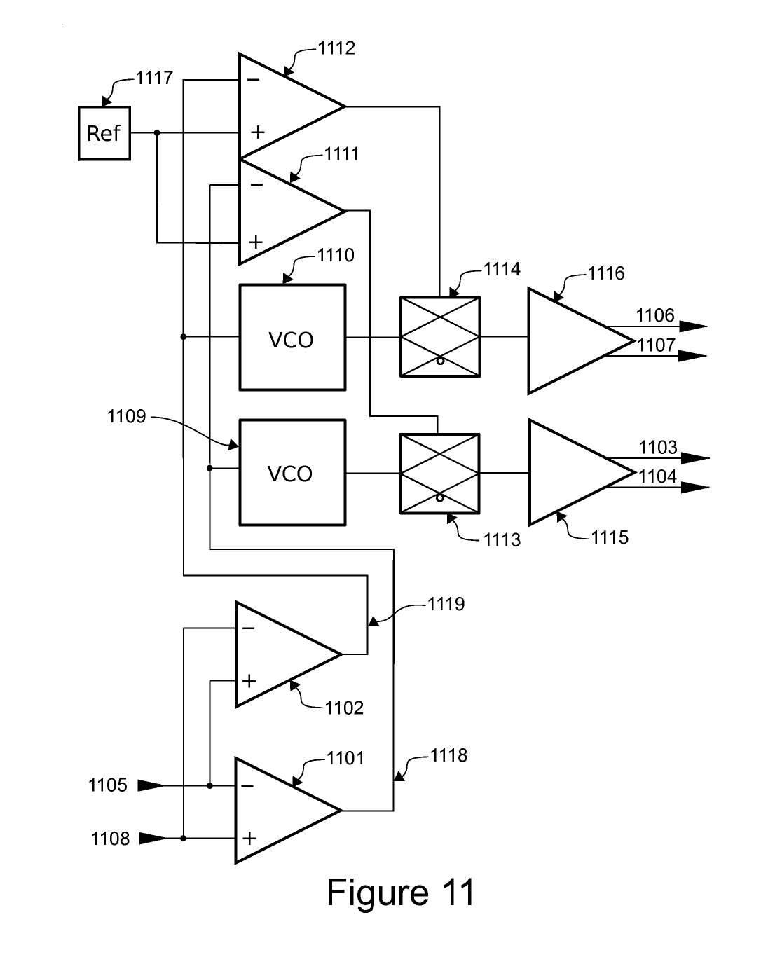

Referring now to FIG. 11, Input 1105 and 1108 refer to signals received by Controller 309 from Sensors 305 and 308, respectively, all of FIG. 3. Differential Current Outputs 1103, 1104, 1106, and 1107 refer to Outputs 303, 304, 306, and 307, respectively, all again of FIG. 3. It is assumed that positive pressures incident upon Sensors 305 and 308 of FIG. 3 will result in positive-going Inputs 1105 and 1108, respectively.

Differential Amplifiers 1101 and 1102 receive Inputs 1105 and 1108 to produce difference Signals 1118 and 1119, respectively. Note that the Output 1118 of Amplifier 1101 will decrease in value in response to Input 1108 being lower than Input 1105, when Sensor 308 receives less internal socket pressure than Sensor 303, both of FIG. 3. Note as well that the Output 1119 of Amplifier 1102 will decrease in value in response to Input 1105 being lower than Input 1108, when Sensor 303 receives less internal socket pressure than Sensor 308, both of FIG. 3.

Amplifier Output 1118 is provided as input to both Voltage Controlled Oscillator 1109 and the inverting input of Comparator 1111. Amplifier Output 1119 is provided as input to both Voltage Controlled Oscillator 1110 and the inverting input of Comparator 1112. It is assumed that Voltage Controlled Oscillators 1109 and 1110 provide outputs of increasing frequency in response to increasing input voltages.

The outputs of Comparators 1111 and 1112 are provided as inputs to the ‘true’ or positive control inputs of Transmission Gates 1113 and 1114, respectively. The outputs of Voltage Controlled Oscillators 1109 and 1110, presumably square wave signals, are provided as input to Transmission Gates 1113 and 1114, respectively. The outputs of Transmission Gates 1113 and 1114 are provided as input to Differential Current Amplifiers 1115 and 1116, respectively. Differential Outputs 1103, 1104, 1106, and 1107 drive Electrodes 303, 304, 306, and 307 of FIG. 3, which correspond to Electrodes 203, 204, 206, and 207 of FIG. 2, as well as Electrodes 103, 104, 106, and 107 of FIG. 1.

Reference 1117 provides a static or dynamic reference value to the non-inverting inputs of Comparators 1111 and 1112. It is assumed that the output value of Reference 1117 corresponds to an input value of Voltage Controlled Oscillators 1109 and 1110 which would cause their output frequencies to be relatively high, at the top of their control ranges. Connected as shown, the output of Comparator 1111 will go high when Amplifier Output 1118 falls below the output of Reference 1117; and the output of Comparator 1112 will go high when Amplifier Output 1119 falls below the output of Reference 1117. In the event that the output of Comparator 1111 is high, it can be seen that the output of Voltage Controlled Oscillator 1109 will be supplied through Transmission Gate 1113 to the input of Differential Amplifier 1115. In the event that the output of Comparator 1112 is high, it can as well be seen that the output of Voltage Controlled Oscillator 1110 will be supplied through Transmission Gate 1114 to the input of Differential Amplifier 1116.

By the connections shown, it can as well be seen that the frequency of Voltage Controlled Oscillator 1109 will decrease when Amplifier Output 1118 decreases, and that the frequency of Voltage Controlled Oscillator 1110 will decrease when Amplifier Output 1119 decreases.

By the discussion above, it can then be seen that Electrodes 1106 and 1107 will be enabled to stimulate tissue when pressure Input 1105 falls below pressure Input 1108 by a defined amount. This corresponds to an internal pressure at Sensor 105 which is less than the pressure at Sensor 108 by a defined amount, both of FIG. 1. It can as well be seen that the frequency of stimulation so enabled to Electrodes 1106 and 1107 will decrease as pressure Input 1105 decreases below pressure Input 1108, hence increasing the period of current pulses supplied as tissue stimulation in response to decreasing differential pressure between Input 1105 and 1108. In that Electrodes 1106 and 1107 correspond to Electrodes 106 and 106, respectively, of FIG. 1, it can therefore be seen that lower pressure at Sensor 105 relative to Sensor 108 will result in higher stimulation current periods enabled and applied to Electrodes 106 and 107, all of FIG. 1. Similarly, it can be seen that lower pressure at Sensor 108 relative 105 will result in higher stimulation current periods enabled and applied to Electrodes 103 and 104, again all of FIG. 1.

The foregoing disclosure describes methods and apparatus whereby a prosthetic socket may be stabilized upon a biological limb without incurring muscular, neurological, or circulatory complications commonly associated with the practice of intentionally wasting residual limb muscle to extend prosthetic socket use. It can be seen that stimulation modalities used herein are not dissimilar to normal muscle activity in a biologically sound limb, yet provide required control of both the socket and air within the socket. Use of the techniques described herein have been shown to improve thermal regulation in a residual limb (a strong indication of improved blood flow) and significantly reduce phantom pain. It is therefore theorized that synchronization of efferent activity with afferent stimulation from normal activity, as afforded by embodiments of the invention, serves to mitigate neurological impact of amputation.

An apparatus for processing instructions may be configured to perform any of the methods described herein. And an apparatus may further include means for performing any of the methods described herein.

Program instructions may be used to cause a general-purpose or special-purpose processing system that is programmed with the instructions to perform the operations described herein. Alternatively, the operations may be performed by specific hardware components that contain hardwired logic for performing the operations, or by any combination of programmed computer components and custom hardware components. The methods described herein may be provided as (a) a computer program product that may include one or more machine readable media having stored thereon instructions that may be used to program a processing system or other electronic device to perform the methods, or (b) at least one storage medium having instructions stored thereon for causing a system to perform the methods. The term “machine readable medium” or “storage medium” used herein shall include any medium that is capable of storing or encoding a sequence of instructions for execution by the machine and that cause the machine to perform any one of the methods described herein. The term “machine readable medium” or “storage medium” shall accordingly include, but not be limited to, memories such as solid-state memories, optical and magnetic disks, read-only memory (ROM), programmable ROM (PROM), erasable PROM (EPROM), electrically EPROM (EEPROM), a disk drive, a floppy disk, a compact disk ROM (CD-ROM), a digital versatile disk (DVD), flash memory, a magneto-optical disk, as well as more exotic mediums such as machine-accessible biological state preserving storage. A medium may include any mechanism for storing, transmitting, or receiving information in a form readable by a machine, and the medium may include medium through which the program code may pass, such as antennas, optical fibers, communications interfaces, etc. Program code may be transmitted in the form of packets, serial data, parallel data, etc., and may be used in a compressed or encrypted format. Furthermore, it is common in the art to speak of software, in one form or another (e.g., program, procedure, process, application, module, logic, and so on) as taking an action or causing a result. Such expressions are merely a shorthand way of stating that the execution of the software by a processing system causes the processor to perform an action or produce a result.

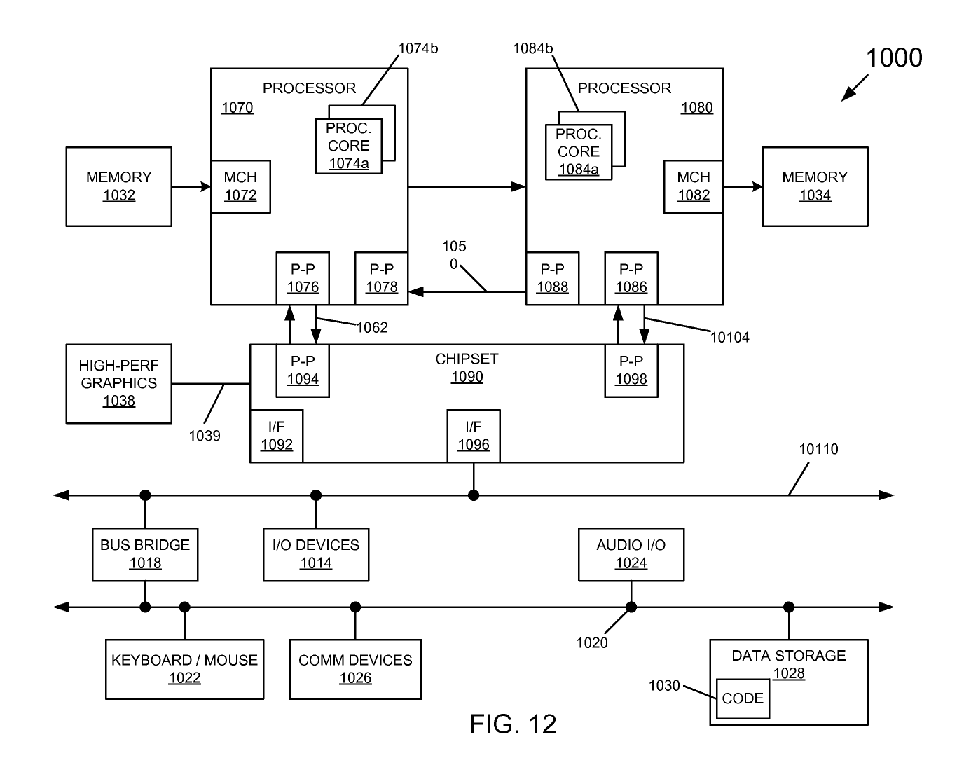

Referring now to FIG. 12, shown is a block diagram of a system embodiment 1000 in accordance with an embodiment of the present invention. Shown is a multiprocessor system 1000 that includes a first processing element 1070 and a second processing element 1080. While two processing elements 1070 and 1080 are shown, it is to be understood that an embodiment of system 1000 may also include only one such processing element. System 1000 is illustrated as a point-to-point interconnect system, wherein the first processing element 1070 and second processing element 1080 are coupled via a point-to-point interconnect 1050. It should be understood that any or all of the interconnects illustrated may be implemented as multi-drop bus rather than point-to-point interconnect. As shown, each of processing elements 1070 and 1080 may be multicore processors, including first and second processor cores (i.e., processor cores 1074a and 1074b and processor cores 1084a and 1084b). Such cores 1074, 1074b, 1084a, 1084b may be configured to execute instruction code in a manner similar to methods discussed herein.

Each processing element 1070, 1080 may include at least one shared cache. The shared cache may store data (e.g., instructions) that are utilized by one or more components of the processor, such as the cores 1074a, 1074b and 1084a, 1084b, respectively. For example, the shared cache may locally cache data stored in a memory 1032, 1034 for faster access by components of the processor. In one or more embodiments, the shared cache may include one or more mid-level caches, such as level 2 (L2), level 3 (L3), level 4 (L4), or other levels of cache, a last level cache (LLC), and/or combinations thereof.

While shown with only two processing elements 1070, 1080, it is to be understood that the scope of the present invention is not so limited. In other embodiments, one or more additional processing elements may be present in a given processor. Alternatively, one or more of processing elements 1070, 1080 may be an element other than a processor, such as an accelerator or a field programmable gate array. For example, additional processing element(s) may include additional processors(s) that are the same as a first processor 1070, additional processor(s) that are heterogeneous or asymmetric to first processor 1070, accelerators (such as, e.g., graphics accelerators or digital signal processing (DSP) units), field programmable gate arrays, or any other processing element. There can be a variety of differences between the processing elements 1070, 1080 in terms of a spectrum of metrics of merit including architectural, microarchitectural, thermal, power consumption characteristics, and the like. These differences may effectively manifest themselves as asymmetry and heterogeneity amongst the processing elements 1070, 1080. For at least one embodiment, the various processing elements 1070, 1080 may reside in the same die package.

First processing element 1070 may further include memory controller logic (MC) 1072 and point-to-point (P-P) interfaces 1076 and 1078. Similarly, second processing element 1080 may include a MC 1082 and P-P interfaces 1086 and 1088. As shown in FIG. 10, MC's 1072 and 1082 couple the processors to respective memories, namely a memory 1032 and a memory 1034, which may be portions of main memory locally attached to the respective processors. While MC logic 1072 and 1082 is illustrated as integrated into the processing elements 1070, 1080, for alternative embodiments the MC logic may be discreet logic outside the processing elements 1070, 1080 rather than integrated therein.

First processing element 1070 and second processing element 1080 may be coupled to an I/O subsystem 1090 via P-P interfaces 1076, 1086 via P-P interconnects 1062, 10104, respectively. As shown, I/O subsystem 1090 includes P-P interfaces 1094 and 1098. Furthermore, I/O subsystem 1090 includes an interface 1092 to couple I/O subsystem 1090 with a high performance graphics engine 1038. In one embodiment, a bus may be used to couple graphics engine 1038 to I/O subsystem 1090. Alternately, a point-to-point interconnect 1039 may couple these components to one another. In an embodiment a bus may be used to couple a TPM or other out-of-band cryptoprocessor (not shown) to I/O subsystem 1090.

In turn, I/O subsystem 1090 may be coupled to a first bus 10110 via an interface 1096. In one embodiment, first bus 10110 may be a Peripheral Component Interconnect (PCI) bus, or a bus such as a PCI Express bus or another third generation I/O interconnect bus, although the scope of the present invention is not so limited.

As shown, various I/O devices 1014, 1024 may be coupled to first bus 10110, along with a bus bridge 1018 which may couple first bus 10110 to a second bus 1020. In one embodiment, second bus 1020 may be a low pin count (LPC) bus. Various devices may be coupled to second bus 1020 including, for example, a keyboard/mouse 1022, communication device(s) 1026 (which may in turn be in communication with a computer network), and a data storage unit 1028 such as a disk drive or other mass storage device which may include code 1030, in one embodiment. The code 1030 may include instructions for performing embodiments of one or more of the methods described above. Further, an audio I/O 1024 may be coupled to second bus 1020.

Note that other embodiments are contemplated. For example, instead of the point-to-point architecture shown, a system may implement a multi-drop bus or another such communication topology. Also, the elements of the Figure may alternatively be partitioned using more or fewer integrated chips than shown in the Figure.

Thus an embodiment resides in the apparatus and technique necessary to stabilize a prosthetic socket upon a residual limb through independent stimulation of multiple enclosed muscle areas. Stimulation intensity for each muscle area is proportionally dominantly controlled by negative differential pressure in a limb area opposing the area to be stimulated. Operation of this proportional control loop may be modified to: Enforce minimum refractory period between stimulations, to allow bloodflow; Enforce maximum time between stimulations, to induce bloodflow during inactivity; and Induce air pumping action, based on gait phase information from accelerometer.

Hardware aspects of embodiments may include electrodes on inner socket surface, selectively embedded conductive material in liner to stimulate underlying muscle, a composite electrode/sensor assembly.

Operational aspects of an embodiment includes decreasing relative pressure at a point in the socket will result in higher stimulation period applied to the opposing side of the socket.

The following is a “section of examples” of embodiments.

An example includes a system for stabilizing a prosthetic socket on a biological limb comprising: a socket to contain the limb; means to measure pressure within at least one area of the socket; and means to stimulate muscle of the contained limb, whereas stimulation intensity is controlled as a function of reduced pressure on the opposing side of the limb.

Another example includes the subject matter of the above examples in the section of examples in addition to further comprising one or more sensors of adequate bandwidth to detect muscle acoustic emissions and calculations necessary to discern muscle contraction force from all other forces incident on the limb.

Another example includes the subject matter of the above examples in the section of examples in addition to wherein calculated muscle contraction force in at least one area is subtracted from the total pressure measured in that area.

In that stimulating muscle within a socket may create additional pressure within the socket that is disrelated to external forces operative on the socket, corruption of pressure measurements within the socket may occur from stimulation, even though negative pressure differential on the side opposing, or furthest from the stimulation area, is explicitly used so as to avoid this corruption. Removal from the total pressure measurement in an area that portion which is known to be resultant of muscle contraction may stabilize overall system control through improving input signal quality.

Another example includes the subject matter of the above examples in the section of examples in addition to wherein modulation of stimulation pulse period is used to vary stimulation intensity in direct relation.

Another example includes the subject matter of the above examples in the section of examples in addition to wherein stimulation is not enabled for periods greater than a specified maximum period.

Another example includes the subject matter of the above examples in the section of examples in addition to wherein a minimum period of no stimulation is enforced between periods of stimulation.

Another example includes the subject matter of the above examples in the section of examples in addition to wherein stimulation periods responsive to internal socket force are modified with gait information obtained from spatial measurements.

Another example includes the subject matter of the above examples in the section of examples in addition to further comprising one or more sensors to detect movement of the prosthesis in space; use of inertial measurements in concert with internal pressure measurements to detect possible air ingress into the socket; and stimulating enclosed limb musculature during periods of possible air ingress.

Another example includes the subject matter of the above examples in the section of examples in addition to wherein stimulation electrodes are laminated or affixed directly to the interior surface of the socket.

Another example includes the subject matter of the above examples in the section of examples in addition to wherein conductive regions of an otherwise non-conductive socket liner transfer current from socket electrodes to limb tissue within the socket liner.

Another example includes the subject matter of the above examples in the section of examples in addition to wherein a force sensor is directly laminated to a stimulation electrode, so as to occupy the same surface area.

Another example includes a method for stabilizing a prosthetic socket on a biological limb comprising: measuring internal pressure at least one location between the limb and internal socket wall; and enabling stimulation to be applied to at least one area of musculature physically opposing an area of lower relative pressure, wherein stimulation intensity so applied is a direct function of lowered relative pressure.

Another example includes the subject matter of the above examples in the section of examples in addition to wherein stimulation intensity is controlled through direct modulation of stimulation pulse period.

Another example includes the subject matter of the above examples in the section of examples in addition to wherein muscle contractile force is differentiated from other forces incident on the socket.

Another example includes the subject matter of the above examples in the section of examples in addition to wherein minimum periods of no stimulation are enforced, so as to allow blood flow.

Another example includes the subject matter of the above examples in the section of examples in addition to wherein maximum periods of stimulation are enforced, so as to allow blood flow.

Another example includes the subject matter of the above examples in the section of examples in addition to wherein stimulation periods responsive to internal socket force are modified with gait information obtained from spatial measurements.

Another example includes the subject matter of the above examples in the section of examples in addition to wherein stimulation electrodes are integrated directly into the inner surface of the prosthetic socket.

Another example includes the subject matter of the above examples in the section of examples in addition to wherein pressure transducers are laminated directly to stimulation electrodes, so as to occupy the same area.

Another example includes the subject matter of the above examples in the section of examples in addition to wherein selective conductive regions of an otherwise non-conductive socket liner are used to conduct current from socket electrodes to the skin of the limb enclosed in the socket liner.

While the present invention has been described with respect to a limited number of embodiments, those skilled in the art will appreciate numerous modifications and variations therefrom. It is intended that the appended claims cover all such modifications and variations as fall within the true spirit and scope of this present invention.

What is claimed is:

1. An orthopedic system comprising:
a socket to contain at least a portion of a patient's limb;
first and second sensors located within the socket, the first sensor located within a first half of the socket and the second sensor located in a second half of the socket;
first and second stimulus electrodes, the first stimulus electrode located in the first half of the socket and the second stimulus electrode located in the second half of the socket; and
a controller to (i) simultaneously sense first pressure corresponding to the first sensor and second pressure corresponding to the second sensor; (2) determine the first pressure is less than the second pressure; (3) stimulate the limb muscle of the contained limb via the first stimulus electrode based on determining the first pressure is less than the second pressure.

2. The system of claim 1, wherein the controller is configured to distinguish between muscle contraction force and the first pressure.

3. The system of claim 1, wherein the first pressure does not include muscle contraction force.

4. The system of claim 1, wherein the controller is configured to determine pressure differential based on the first and second pressures and stimulate the limb muscle via the first stimulus electrode based on determining the pressure differential.

5. The system of claim 1, wherein determining the first pressure is less than the second pressure includes determining a pressure differential based on the first and second pressures.

6. The system of claim 5, wherein stimulating the limb muscle via the first stimulus electrode includes lengthening stimulation pulse width as the pressure differential increases and decreasing stimulation pulse width as the pressure differential decreases.

7. The system of claim 1, wherein the controller is to detect movement of the socket in space and stimulate the limb during the application of propulsive forces to the socket and continue to stimulate the limb after the application of propulsive forces to the socket discontinue to disallow air ingress into the socket as axial force on the socket plummets.

8. The system of claim 1, wherein the controller includes a processor coupled to a memory.

9. The system of claim 1, wherein the controller is to detect movement of the socket in space and (i) stimulate the limb during the application of propulsive forces to the socket and continue to stimulate the limb after the application of propulsive forces to the socket discontinue to disallow air ingress into the socket as axial force on the socket plummets, and (ii) provide no stimulation to the limb, after stimulating the limb, to allow air to exit the socket.

10. The system of claim 1, wherein the controller is to detect movement of the socket in space and alternately stimulate and not stimulate the limb to induce a pumping cycle to retain a socket vacuum.

11. The system of claim 1 first and second stimulus electrodes are laminated and affixed directly to an interior surface of the socket.

12. The system of claim 1 comprising a non-conductive socket line with conductive regions that transfer current from the first electrode to the limb.

13. The system of claim 1, wherein the first sensor is directly laminated to the first electrode so as to occupy the same surface area.

14. The system of claim 1, wherein the controller is to not stimulate the limb via the second stimulus electrode when stimulating the limb via the first stimulus electrode based on determining the first pressure is less than the second pressure.
